(19) United States
(12) Patent Application Publication
(10) Pub. No.: US 2026/0199361 A1
(43) Pub. Date: Jul. 16, 2026

(54) COMPOSITIONS COMPRISING PRALATREXATE FOR SUBCUTANEOUS ADMINISTRATION AND METHODS OF USE THEREOF

(71) Applicant: ACROTECH BIOPHARMA INC, East Windsor, NJ (US)

(72) Inventors: Jami DILEEP, Hyderabad (IN); Madduru DASTAGIRI, Hyderabad (IN); Ashish ANVEKAR, East Windsor, NJ (US); Arun JANA, East Windsor, NJ (US); Nagaprasad VISHNUBHOTLA, East Windsor, NJ (US); Meenakshisunderam SIVAKUMARAN, East Windsor, NJ (US)

(73) Assignee: ACROTECH BIOPHARMA INC, East Windsor, NJ (US)

(21) Appl. No.: 19/562,920

(22) Filed: Mar. 11, 2026

Related U.S. Application Data
(63) Continuation of application No. PCT/US2024/046740, filed on Sep. 13, 2024.
(60) Provisional application No. 63/538,720, filed on Sep. 15, 2023.

(51) Int. Cl. A61K 31/519; A61K 9/00; A61K 47/02; A61K 47/26

(52) U.S. Cl. CPC A61K 31/519; A61K 9/0019; A61K 47/02; A61K 47/26

(57) ABSTRACT

The present disclosure provides compositions comprising Pralatrexate for subcutaneous administration. The present disclosure also provides methods of administering the compositions comprising Pralatrexate, as disclosed herein, for the treatment of disease (e.g., lymphoma).

### CROSS REFERENCE TO RELATED APPLICATIONS

The present application claims priority to and the benefit of U.S. App. No. 63/538,720, filed Sep. 15, 2023, which is hereby incorporated by reference in its entirety.

### BACKGROUND

Lymphoma is a group of blood and lymph tumors that develop from lymphocytes (a type of white blood cell).

Pralatrexate is the first drug approved as a treatment for patients with relapsed or refractory peripheral T-cell lymphoma, or PTCL—a biologically diverse group of aggressive blood cancers. Pralatrexate is currently marketed under the trade name FOLOTYN®. The recommended dosage of FOLOTYN® is 30 mg/m² intravenously over 3-5 minutes once weekly for 6 weeks in 7-week cycles until progressive disease or unacceptable toxicity. It is supplied as either 20 mg (1 mL) or 40 mg (2 mL) single-dose vials at a concentration of 20 mg/mL. FOLOTYN® is administered in an undiluted form by aseptically withdrawing the calculated dose from the appropriate number of vial(s) into a syringe via the side port of a free-flowing 0.9% Sodium Chloride Injection intravenously over 3-5 minutes. For an adult patient possessing body surface area of 1.9 m² at least two or three vials need to be administered to reach the required dose. This method of administration leads to patient inconvenience. This also increases the cost of the treatment, as it always requires administration in a hospital set up.

Further IV injections are given either as a 1-to-2-min-bolus or an infusion with a longer duration to facilitate administering precise doses in a controlled manner or in providing a rapid response. IV dosing requires the skilled insertion of a needle or catheter directly into a vein, a process that can be challenging, especially in certain patient types such as obese, palliative-care, and neonate patients. Direct IV access also carries a risk of systemic infection. IV drugs are administered by trained medical personnel in a hospital or physician's office and, because of the rapid effects achieved, patients are typically observed for undesired side effects following injection. This entire process occupies both space in a hospital and specialized nursing time.

Therefore, a need exists for development of ready to use pharmaceutical compositions containing Pralatrexate that are stable in solution, contain a sufficient concentration of Pralatrexate, and that can be administered without the need of hospital set up.

### SUMMARY OF THE INVENTION

The disclosure provides compositions comprising Pralatrexate for subcutaneous administration and methods of use thereof.

In an aspect, the present disclosure provides a pharmaceutical composition including Pralatrexate or a pharmaceutically acceptable salt, hydrate or ester thereof, for subcutaneous administration. The pharmaceutical composition may be isoosmotic and may have a pH between about 7 to about 8.5.

In another aspect, the present disclosure provides a method for treating peripheral T-cell lymphoma involving subcutaneous administration of a subcutaneous pharmaceutical composition of Pralatrexate to a subject in need thereof. The pharmaceutical composition is isoosmotic and has a pH between about 7 to about 8.5.

In any of the above aspects, or embodiments thereof, the composition includes one or more pharmaceutically acceptable excipients.

In any of the above aspects, or embodiments thereof, the composition includes at least about 30 mg/ml or at least about 40 mg/ml of Pralatrexate. For example the composition may comprise at most about 100 mg/ml Pralatrexate. In various implementaytions, the composition comprises from about 30 mg/ml to 90 mg/ml Pralatrexate, from about 30 mg/ml to about 80 mg/ml Pralatrexate, from about 30 mg/ml to about 70 mg/ml Pralatrexate, from about 35 mg/ml to 90 mg/ml Pralatrexate, from about 35 mg/ml to about 80 mg/ml Pralatrexate, from about 35 mg/ml to about 70 mg/ml Pralatrexate, from about 40 mg/ml to 90 mg/ml Pralatrexate, from about 40 mg/ml to about 80 mg/ml Pralatrexate, or from about 40 mg/ml to about 70 mg/ml Pralatrexate. In certain aspects, the composition comprises about 40 mg/ml Pralatrexate, about 50 mg/ml Pralatrexate, about 60 mg/ml Pralatrexate, about 70 mg/ml Pralatrexate, or about 80 mg/ml Pralatrexate. In some embodiments, the composition comprises about 60 mg/ml Pralatrexate.

In any of the above aspects, or embodiments thereof, the composition includes a pharmaceutically acceptable alkalizer, solubilizer, surfactant, and/or osmogen.

In any of the above aspects, or embodiments thereof, the excipient is one or more of a buffer, a pH stabilizer, an isotonicity agent, an antioxidant, a preservative, and a solubility and/or viscosity-enhancing agent.

In any of the above aspects, or embodiments thereof, the alkalizer is lithium hydroxide, sodium hydroxide, potassium hydroxide or calcium hydroxide.

In any of the above aspects, or embodiments thereof, the solubilizer is one or more of propylene glycol, polyethylene glycol 300/400, glycerin, ethanol, polysorbate 80, Cremophor EL, N-methyl-2-pyrrolidone (NMP), or meglumine.

In any of the above aspects, or embodiments thereof, the surfactant is Tween 80 or meglumine.

In any of the above aspects, or embodiments thereof, the osmogen is one or more of potassium chloride, sodium chloride, or mannitol.

In any of the above aspects, or embodiments thereof, the composition has an osmolarity of between about 250 mOsm to about 350 mOsm.

In any of the above aspects, or embodiments, thereof, the composition has greater bioavailability of pralatrexate when administered to a subject as compared to an intravenous formulation of Pralatrexate (e.g., as determined by comparing AUC_{0→τ}, AUC_{0→24}, or AUC_{0→inf} values following administration of intravenous or subcutaneous injections of the same weight of Pralatrexate such as 5 mg Pralatrexate). In certain aspects, the composition has at least about 10% or at least about 20% or at least about 30% greater bioavailability of Pralatrexate as compared to an intravenous formulation of Pralatrexate.

In any of the above aspects, or embodiments thereof, a single dose subcutaneous administration of the composition to a subject may provide at least one of:
a) a Cₘₐₓ of from 60 to 120 ng/ml,
b) an AUC_{0→τ} of from 150 to 270 hr*ng/ml,
c) an AUC_{0→24} of from 180 to 300 hr*ng/ml,
d) an AUC_{0→inf} of from 150 to 400 hr*ng/mL,
e) a Tₘₐₓ of from 0.5-1.5 hr,
f), a Kₑₗ of from 0.2-0.4 l/hr,
g) a t_{1/2} of from 1.3-6.6 hr, and
h) a mean subcutaneous absolute bioavailability of from 1.7-2.1.

For example, a single dose of subcutaneous administration of the composition to a subject provides a Cₘₐₓ of from 60 to 120 ng/ml and a Tₘₐₓ of from 0.5-1.5 hr. In some embodiments, the a single dose of subcutaneous administration of the composition to a subject provides an AUC_{0→24} of from 180 to 300 hr*ng/ml.

In any of the above aspects, or embodiments thereof, the pharmaceutical composition may comprise one or more of:
a) from about 30 mg/ml to about 110 mg/ml Pralatrexate;
b) a pH adjuster (e.g., hydrochloric acid);
c) optionally from about 5 mg/ml to about 20 mg/ml alkalizer (e.g., sodium hydroxide,
d) optionally from about 5 mg/ml to about 35 mg/ml buffer (e.g., sodium bicarbonate, potassium dihydrogen phosphate);
e) optionally from about 0.2 mg/ml to about 10 mg/ml osmogene (e.g., sodium chloride, potassium chloride, mannitol) or from about 6 to about 10 mg/ml non-inorganic salt osmogene (e.g., mannitol); and
f) optionally from about 40 mg/ml to about 110 mg/ml surfactant (e.g., meglumine, Tween 80).
   The pharmaceutical composition may comprise one or more of:
a) from about 30 mg/ml to about 110 mg/ml Pralatrexate;
b) hydrochloric acid;
c) optionally from about 5 mg/ml to about 20 mg/ml sodium hydroxide,
d) optionally from about 5 mg/ml to about 35 mg/ml sodium bicarbonate, potassium dihydrogen phosphate, or a combination thereof;
e) optionally from about 0.2 mg/ml to about 10 mg/ml osmogen selected from sodium chloride, potassium chloride, mannitol or from about 6 to about 10 mg/ml mannitol; and
f) optionally from about 40 mg/ml to about 110 mg/ml surfactant of a surfactant selected from meglumine and Tween 80.

Compositions and articles defined by the invention were isolated or otherwise manufactured or tested in connection with the examples provided below. Other features and advantages of the invention will be apparent from the detailed description, and from the claims.

### Definitions

Unless defined otherwise, all technical and scientific terms used herein have the meaning commonly understood by a person skilled in the art to which this invention belongs. The following references provide one of skill with a general definition of many of the terms used in this invention: Singleton et al., Dictionary of Microbiology and Molecular Biology (2nd ed. 1994); The Cambridge Dictionary of Science and Technology (Walker ed., 1988); The Glossary of Genetics, 5th Ed., R. Rieger et al. (eds.), Springer Verlag (1991); and Hale & Marham, The Harper Collins Dictionary of Biology (1991). As used herein, the following terms have the meanings ascribed to them below, unless specified otherwise.

By “agent” is meant a peptide, nucleic acid molecule, or small compound. In some embodiments, the agent is Pralatrexate.

By “alkalizer” is meant a basic compound which raises the pH when added to a solution. In some embodiments, the alkalizer is lithium hydroxide, sodium hydroxide, potassium hydroxide or calcium hydroxide.

By “ameliorate” is meant decrease, suppress, attenuate, diminish, arrest, or stabilize the development or progression of a disease. In some embodiments, the amelioration is a decrease in the number of neoplastic cells, such as lymphoma cells, a suppression of the growth of neoplastic cells, such as lymphoma cells, or the stabilization of the progression of a neoplasia, such as a lymphoma.

By “analog” is meant a molecule that is not identical, but has analogous functional or structural features. For example, a chemical analog has a structure similar to that of a given chemical, but with differences in one or more atoms, functional groups, or substructures, which are replaced with other atoms, groups, or substructures, while retaining the biological activity of the given chemical.

As used herein, “API stable” refers to less than or equal to about a ±10% variation in the amount of active pharmaceutical ingredients (API), for example, Pralatrexate, in a solution, for example, a pharmaceutical composition, over time. In various embodiments, API stable can refer to less than or equal to about a ±7.5% variation in the amount of API in a solution over time. In some embodiments, API stable can refer to less than or equal to about a ±5% variation in the amount of API in a solution over time, In certain embodiments, API stable can refer to less than or equal to about a ±3% variation in the amount of API in a solution over time. In particular embodiments, API stable can refer to less than or equal to about a ±2% variation, or a ±1% variation, in the amount of API in a solution over time.

As used herein, a “buffer” refers to an aqueous solution that is resistant to changes in pH. A buffer can include a weak acid and its salt, or a weak base and its salt, which assist in maintaining the stability of the pH. Examples of buffers used in pharmaceutical compositions include bicarbonate buffers, carbonate buffers, citrate buffers, histidine buffers, phosphate buffers, tartrate buffers, and combinations thereof. Certain of these buffers are suitable for pharmaceutical compositions administered subcutaneously.

In this disclosure, “comprises,” “comprising,” “containing” and “having” and the like can have the meaning ascribed to them in U.S. Patent law and can mean “includes,” “including,” and the like; “consisting essentially of” or “consists essentially” likewise has the meaning ascribed in U.S. Patent law and the term is open-ended, allowing for the presence of more than that which is recited so long as basic or novel characteristics of that which is recited is not changed by the presence of more than that which is recited, but excludes prior art embodiments.

By “decreases” is meant a reduction by at least about 5% relative to a reference level. A decrease may be by 5%, 10%, 15%, 20%, 25% or 50%, or even by as much as 75%, 85%, 95% or more and any intervening percentages.

By “disease” is meant any condition or disorder that damages or interferes with the normal function of a cell, tissue, or organ. In some embodiments, the disease is a lymphoma, such as Non-Hodgkins Lymphoma (NHL); diffuse large B-cell lymphoma (DLBCL); follicular lymphoma (FL); Hodgkin's Disease; Burkitt's Lymphoma; cutaneous T cell lymphoma; primary central nervous system lymphoma, lymphomatous metastases, T-cell lymphomas, and B-cell lymphomas. In some embodiments, the disease is peripheral T cell lymphoma.

By “effective amount” is meant the amount of a composition of the present disclosure required to ameliorate the symptoms of a disease relative to an untreated patient. The effective amount of active compound(s) used to practice the present invention for therapeutic treatment of a disease varies depending upon the manner of administration, the age, body weight, and general health of the subject. Ultimately, the attending physician or veterinarian will decide the appropriate amount and dosage regimen. Such amount is referred to as an “effective” amount. In some embodiments, the effective amount is the amount of a composition of the present disclosure required to ameliorate the symptoms of a lymphoma, such as a peripheral T cell lymphoma, in a patient, relative to an untreated patient.

By “ester” is meant is a compound in which the hydrogen atom (H) of at least one hydroxyl group (—OH) is replaced by an organyl group (—R) such as a lower alkyl (e.g., C₁-C₄ alkyl group).

By “hydrate” is meant a compound containing water bound to the structure of another molecule. Hydrates may formed through the addition of water to another chemical (e.g., Pralatrexate).

As used herein, an “isoosmotic solution,” an “isoosmotic composition,” an “isoosmotic pharmaceutical composition,” or a pharmaceutical composition that is “isoosmotic” refers to a solution or a composition that has the same or similar concentration of solutes as found in bodily fluids. In certain embodiments, a pharmaceutical composition that is “isoosmotic” can have an osmolarity in the range of about 250 mOsM to about 350 mOsM or when the osmolality of the composition is in the range of about 250 mOsm/kg to about 350 mOsm/kg.

By “lymphoma” is meant a neoplasia involving the lymphatic system. For example, lymphomas include, but are not limited to, Non-Hodgkins Lymphoma (NHL); diffuse large B-cell lymphoma (DLBCL); follicular lymphoma (FL); Hodgkin's Disease; Burkitt's Lymphoma; cutaneous T cell lymphoma; primary central nervous system lymphoma, and lymphomatous metastases. In most cases, lymphoma is characterized by the presence of cancerous B cells. However, in T cell lymphomas, the disease state is characterized by cancerous T lymphocytes.

T-cell lymphomas are lymphomas in which the T cells of the patient are determined to be cancerous. T-cell lymphomas encompass a variety of conditions including, without limitation:
(a) lymphoblastic lymphomas in which the malignancy occurs in primitive lymphoid progenitors from the thymus;
(b) mature or peripheral T cell neoplasms, including T cell prolymphocytic leukemia, T-cell granular lymphocytic leukemia, aggressive NK-cell leukemia, cutaneous T cell lymphoma (Mycosis fungoides/Sezary syndrome), anaplastic large cell lymphoma, T cell type, enteropathy-type T cell lymphoma, Adult T-cell leukemia/lymphoma including those associated with HTLV-1, and angioimmunoblastic T cell lymphoma, and subcutaneous panniculitic T cell lymphoma; and
(c) peripheral T cell lymphomas (PTCL) that initially involve a lymph node paracortex and never grow into a true follicular pattern.

Treatment approaches for PTCL have mirrored diffuse large B-cell lymphoma, although the majority of PTCLs have an inferior prognosis compared with their B-cell counterparts.

As used herein, “obtaining” as in “obtaining an agent” includes synthesizing, purchasing, or otherwise acquiring the agent.

By “osmogen” is meant a compound which dissolves in fluid to produce osmotic pressure. Osmogens are often found as a key ingredient in osmotic pharmaceutical formulations, such as osmotic pump formulations. In such formulations, upon penetration of biological fluid into the osmotic pump through semipermeable membrane, osmogens are dissolved in the biological fluid, which creates osmotic pressure buildup inside the pump and pushes medicament outside the pump through delivery orifice. Osmogens include inorganic salts and carbohydrates. In some embodiments, the osmogene is one or more of potassium chloride, sodium chloride, or mannitol.

As used herein, “osmoticity” and “osmolality” refer to the osmotic pressure of a solution such as a pharmaceutical composition. Osmoticity often is measured in osmolarity (“Osm/L” or “OsM”) or osmolality (“Gsm/kg”), which can be used interchangeably herein. When measuring freezing point depression, the observed value is the osmolality of the solution. In contrast to tonicity, osmoticity accounts for un-ionized solutes in a solution such that when present, the osmolarity or osmolality of the solution will be higher than its tonicity.

As used herein, “pharmaceutically acceptable” refers to a substance that is acceptable for use in pharmaceutical applications from a toxicological perspective and does not adversely interact with the active ingredient. Accordingly, pharmaceutically acceptable carriers are those that are compatible with the other ingredients in the composition and are biologically acceptable. Supplementary active ingredients can also be incorporated into the pharmaceutical compositions.

As used herein, “pH stable” refers to less than or equal to about a ±0.5 pH value variation in the pH of a solution, for example, a pharmaceutical composition, over time. In various embodiments, pH stable can refer to less than or equal to about a ±0.4 pH value variation in the pH of a solution over time. In some embodiments, pH stable can refer to less than or equal to about a ±0.3 pH value variation in the pH of a solution over time, in certain embodiments, pH stable can refer to less than or equal to about a ±0.2 pH value variation in the pH of a solution over tune. In particular embodiments, pH stable can refer to less than or equal to about a ±0, 1 pH value variation in the pH of a solution over time.

As used herein, “physiological pH” refers to a pH of about 7.4.

By “pralatrexate/Pralatrexate” is meant “(2S)-24[4-[(1RS)-1-[(2, 4-diaminopteridin-6-yl)methyl] but-3-ynyl]benzoyl]-amino]pentanedioic acid”, “10-Propargy 1-10-deazaminopterin”, “PDX”, or a compound having the following structure:

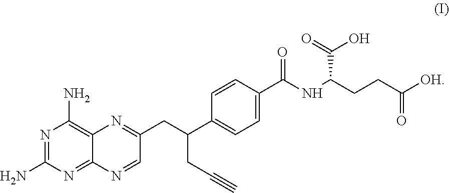 [SMILES: C#CCC(Cc1cnc2nc(N)nc(N)c2n1)c1ccc(C(=O)N[C@@H](CCC(=O)O)C(=O)O)cc1]

By “reduces” is meant a negative alteration of at least 10%, 25%, 50%, 75%, or 100%.

By “reference” is meant a standard or control condition or a standard or control subject. In some embodiments, the reference is an untreated subject having a disease. In some embodiments, the reference is an untreated lymphoma, or neoplastic lymphatic cell, such as a T-cell or B-cell. In some embodiments, the reference is a healthy subject. In some embodiments, the reference is a healthy lymphatic cell.

By “salt” is meant an ionizable compound which has been combined with a counter-ion to form a neutral complex.

By “solubilizer” is meant a compound which increases the solubility of a substance. Solubilizers may also be surfactants and vice-versa. In some embodiments, the solubilizer is one or more of propylene glycol, polyethylene glycol 300/400, glycerin, ethanol, polysorbate 80, Cremophor EL, N-methyl-2-pyrrolidone (NMP), or meglumine.

By “subject” is meant a mammal, including, but not limited to, a human or non-human mammal, such as a bovine, equine, canine, ovine, or feline.

By “surfactant” is meant a compound which decreases the surface tension of a liquid. Surfactants may function as emulsifiers, wetting agents, detergents, foaming agents, or dispersants. In some embodiments, the surfactant is Tween 80 and/or meglumine.

Ranges provided herein are understood to be shorthand for all of the values within the range. For example, a range of 1 to 50 is understood to include any number, combination of numbers, or sub-range from the group consisting 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50.

As used herein, “tonicity” refers to the ionic strength or concentration of ions in a solution such as a pharmaceutical composition. Tonicity often is measured in molarity (“M”). As used herein, an “isotonic solution,” an “isotonic composition,” an “isotonic pharmaceutical composition,” and a pharmaceutical composition that is “isotonic” refers to a solution or composition that has the same or similar concentration of ions as found in bodily fluids.

As used herein, the terms “treat,” treating,” “treatment,” and the like refer to reducing or ameliorating a disorder and/or symptoms associated therewith. It will be appreciated that, although not precluded, treating a disorder or condition does not require that the disorder, condition or symptoms associated therewith be completely eliminated. In some embodiments, the treatment is the amelioration of one or more symptoms of a neoplasia, such as a lymphoma. In some embodiments, the treatment results in an increase in average lifespan in a treated subject, relative to a reference (e.g., an untreated, diseased subject).

Unless specifically stated or obvious from context, as used herein, the term “or” is understood to be inclusive. Unless specifically stated or obvious from context, as used herein, the terms “a”, “an”, and “the” are understood to be singular or plural.

Unless specifically stated or obvious from context, as used herein, the term “about” is understood as within a range of normal tolerance in the art, for example within 2 standard deviations of the mean. About can be understood as within 10%, 9%, 8%, 7%, 6%, 5%, 4%, 3%, 2%, 1%, 0.5%, 0.1%, 0.05%, or 0.01% of the stated value. Unless otherwise clear from context, all numerical values provided herein are modified by the term about.

The recitation of a listing of chemical groups in any definition of a variable herein includes definitions of that variable as any single group or combination of listed groups. The recitation of an embodiment for a variable or aspect herein includes that embodiment as any single embodiment or in combination with any other embodiments or portions thereof.

Any compositions or methods provided herein can be combined with one or more of any of the other compositions and methods provided herein.

### DETAILED DESCRIPTION OF THE INVENTION

The present disclosure features compositions comprising Pralatrexate for subcutaneous administration. The present disclosure also provides ready to use stable pharmaceutical compositions comprising Pralatrexate for subcutaneous administration. The present disclosure also provides methods of administering the compositions comprising Pralatrexate, as disclosed herein, for the treatment of lymphoma.

In some embodiments, provided herein is a concentrated ready to use stable pharmaceutical composition of Pralatrexate, for example, containing at least about 40 mg/mL, 50 mg/mL, 60 mg/mL, 80 mg/ml or 100 mg/ml of Pralatrexate which can be administered subcutaneously without the need of hospital set up. In some embodiments, the compositions comprising Pralatrexate, as disclosed herein, increase compliance in the subject to whom the compositions are administered, as compared to a reference subject being administered a standard or conventional formulation of Pralatrexate.

In some embodiments, the present disclosure provides ready to use stable Pralatrexate compositions which do not include using diluents like 0.9% Sodium Chloride Injection, and methods of administration thereof.

In some embodiments, the present disclosure provides ready to use stable Pralatrexate compositions formulated for administration in a simple manner without hospital set up, and methods of administration thereof.

### Pralatrexate

Pralatrexate (also known as PDX) was first disclosed in Journal of Medicinal Chemistry. 36:2228-2231 (1993) by DeGraw et al., and subsequently in U.S. Pat. Nos. 5,374,726, 5,354,741 and 6,028,071, each of which are hereby incorporated by reference. It is represented by following structure.

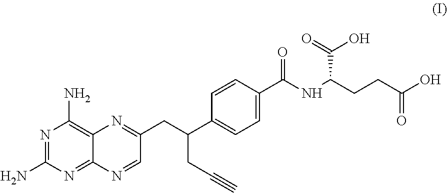 [SMILES: C#CCC(Cc1cnc2nc(N)nc(N)c2n1)c1ccc(C(=O)N[C@@H](CCC(=O)O)C(=O)O)cc1]

Pralatrexate exerts anti-folate activity via the inhibition of dihydrofolate reductase (DHFR). Pralatrexate has high affinity for the reduced folate carrier 1 (RFC-1) protein and is an efficient substrate for polyglutamylation by the enzyme folypolyglutamyl synthetase (FPGS). It has been tested and found useful in the treatment of cancer. In its racemic form, (2S)-24[4-[(1RS)-1-[(2,4-diaminopteridin-6-yl)methyl] but-3-ynylThenzoyl]amino]-pentanedioic acid has been approved by the U.S. Food and Drug Administration (FDA) as a treatment for relapsed and refractory peripheral T-cell lymphoma.

Futher, U.S. Pat. Nos. 7,622,470 and 8,299,078, each of which are hereby incorporated by reference, disclose a method for treatment of peripheral T cell lymphoma excluding mycosis fungoides comprising administering to a human having a peripheral T cell lymphoma other than mycosis fungoides a composition comprising a therapeutically effective amount of Pralatrexate.

The 10-deazaaminopterin compound of the current disclosure (e.g., 10-propargyl-10-deazaaminopterin, also known as Pralatrexate) can be formulated in the form of an acid addition salt. These salts are formed with one or more free NH₂ groups of the heteroaroyl-10-deazaaminopterin molecule. Typically, the compounds are injected in the form of their sodium salts in aqueous solution. Other salts, such as K, Ca, NH₄, and the like, could be used as prepared from the appropriate hydroxide or carbonates.

The acid addition salts are the pharmaceutically acceptable, nontoxic addition salts with suitable acids, such as those with inorganic acids, for example, hydrochloric, hydrobromic, nitric, sulphuric, and phosphoric acids, and with organic acids, such as organic carboxylic acids, for example, glycolic, maleic, hydroxymaleic, malic, tartaric, citric, salicylic, acetyloxybenzoic, nicotinic, and isonicotinic acid, and organic sulphonic acids, for example, methanesulphonic, ethanesulphonic, 2-hydroxyethanesulphonic, toluene-p-sulphonic, and naphthalene-2-sulphonic acid.

An acid addition salt can be converted into the free compound according to known methods, for example, by treating it with a base, such as with a metal hydroxide or alkoxide, for example, an alkali metal or alkaline earth metal hydroxide, for example, lithium hydroxide, sodium hydroxide, potassium hydroxide or calcium hydroxide; with a metal carbonate, such as an alkali metal or an alkaline earth metal carbonate or hydrogen carbonate, for example, sodium, potassium or calcium carbonate or hydrogen carbonate, with ammonia; or with a hydroxyl ion exchange resin, or with any other suitable reagent. An acid addition salt may also be converted into another acid addition salt according to known methods, for example, a salt with an inorganic acid may be treated with a metal salt, for example a sodium, barium or silver salt, of an acid in a suitable diluent, in which a resulting inorganic salt is insoluble and is thus removed from the reaction medium. An acid-addition salt may also be converted into another acid addition salt by treatment with an anion exchange preparation.

### Combination Therapies

Further, Pralatrexate may be used in combinations with other cytotoxic and antitumor compounds, including vinca alkaloids such as vinblastine, navelbine and vindesine; probenicid, nucleotide analogs such as gemcitabine, 5-fluorouracil, and cytarabine; alkylating agents such as cyclophosphamide or ifosfamide; cisplatin or carboplatin; leucovorin; taxanes such a paclitaxel or docetaxel; anti-CD20 monoclonal antibodies, with or without radioisotopes, and antibiotics such as doxorubicin and mitomycin. Combinations of Pralatrexate with several of these other antitumor agents or with growth factor inhibitors and anti-angiogenic agents may also be used.

Pralatrexate and other agents may be concurrently administered or utilized in combination as part of a common treatment regimen, in which the Pralatrexate and the other agent(s) are administered at different times. For example, the other agent may be administered before, immediately afterward or after a period of time (for example 24 hours) relative to the Pralatrexate administration. Thus, for purposes of this application, the term administering refers generally to concurrent administration or to sequential administration of the drugs and in either order in a parallel treatment regimen with or without a separation in time between the drugs unless otherwise specified.

Pralatrexate is suitably used in combination with folic acid and vitamin B12 supplementation to reduce the side effects of the treatment. For example, patients may be treated with folic acid (1 mg/m² daily starting 1 week prior to treatment with Pralatrexate) and B12 (1 mg/m² monthly).

### Pharmaceutical Compositions

The present invention relates to ready to use stable pharmaceutical compositions comprising a high concentration of Pralatrexate for subcutaneous administration.

For subcutaneous (SC) administration, discomfort and pain during administration should be minimized so as to avoid poor patient compliance with the treatment regimen. Factors that can contribute to pain and discomfort perceived by a patient upon, during, or after subcutaneous administration include the injection volume, the pH of the composition, and the osmoticity or tonicity of the composition. Moreover, such a composition should be stable in solution so that it readily is available for use and/or can be pre-loaded into a variety of dispensing devices.

During SC administration, a needle is inserted through the epidermal and dermal layers of the skin and into the fatty subcutaneous tissue. Following injection, the drug reaches the bloodstream via the capillaries or the lymphatic system, depending on the molecular size of the therapeutic agent.

SC injections have several immediate advantages over other injection types. In contrast to the skilled personnel required for the administration of intravenous (IV) and intramuscular (IM) injections, SC injections can be administered by the patient. A small needle is required (length of ⅜-⅝ in.), and the injections are not generally painful and carry a reduced risk of infection and other complications. If an infectious agent is injected subcutaneously, it is generally limited to a local infection rather than a systemic infection. For patients requiring multiple doses, SC injections offer a broader range of alternative sites. The resultant drug absorption is also slower than IV and may avoid the risks of bolus administration.

From many perspectives, including reduced pain, improved patient quality of life, reduced cost of patient care, and reduced risk of infection, SC represents a preferred route for administering a drug by self-injection. Many drugs, including insulin and heparin, have been delivered subcutaneously for many years with excellent outcomes.

The recommended dose of commercially available Pralatrexate (FOLOTYN®) is 30 mg/m² administered as an intravenous push over 3-5 minutes via the side port of a free-flowing 0.9% Sodium Chloride Injection, intravenous line once weekly for 6 weeks in 7-week cycles until progressive disease or unacceptable toxicity. The calculated dose of Pralatrexate should be aseptically withdrawn into a syringe for immediate use. The required dose of Pralatrexate is typically based on body surface area of the patient which is around 1.9 m². Therefore at least two to three injection is required to achieve the targeted dose.

The present invention provides compositions for the subcutaneous administration of Pralatrexate. More specifically, the present invention provides a stable and concentrated Pralatrexate composition which can be administered subcutaneously. Such pharmaceutical compositions can be useful in the treatment of for treatment of peripheral t-cell lymphoma.

Development of subcutaneous dosage form involves selection of at least one of buffer, pH, stabilizers, isotonicity agents, antioxidants, preservatives, and solubility and/or viscosity-enhancing agents. The pH is one of the critical aspects of parenteral composition, which should have a target pH as much as possible close to physiological pH. A pharmaceutical composition for subcutaneous administration requires a pH at about or at physiological pH, or be within a relatively narrow range of pH in near physiological pH (e.g., between 6 or 6.5 to about 8.5) so that the administered composition readily can equilibrate to physiological pH. The pharmaceutical composition of the present invention for subcutaneous administration need to be substantially isoosmotic or isotonic.

The acceptable range is pH 2-11 for intravenous and intramuscular injection and pH 4-9 for subcutaneous injection due to potential irritation issue. The pH of the solution can be adjusted by using acids and bases (alkalizer or pH adjuster) without buffer due to in vivo tolerability considerations.

In certain embodiments, the pharmaceutical composition can have a pH in the range of about 7.2 to about 8, about 7.2 to about 7.8, or about 7.2 to about 7.6, in particular embodiments, the pharmaceutical compositions can have a pH in the range of about 7.3 to about 8, about 7.3 to about 7.8, about 7.3 to about 7.6, or about 7.3 to about 7.5. In some embodiments, the pharmaceutical composition can have a pH in the range of about 7.4 to about 8, about 7.4 to about 7.8, or about 7.4 to 7.6.

Parenteral compositions should be ideally isotonic whenever possible. Dextrose, Mannitol, glycerol, and sodium chloride are the most commonly used agents to adjust the tonicity of a solution composition. Hypertonic solutions may cause blood cell deformation, whereas a hypotonic solution may lead to rupture of blood cell that results in hemolysis. Compositions with an osmotic value in the range of 250 to 350 mOsm are acceptable. The preferred osmotic agents may be selected from sodium chloride, potassium chloride, mannitol or dextrose.

In various embodiments, the pharmaceutical composition have an osmolality of between about 250 mOsM (or 250 mOsm/kg) to about 350 mOsM (or 350 mOsm/kg), about 275 mOsM (or 275 mOsm/kg) to about 325 mOsM (or 325 mOsm/kg), or about 290 mOsM (or 290 mOsm/kg) to about 310 mOsM (or 310 mOsm/kg).

In addition to alteration of pH, solubilizers, such as surfactants, solvents, co-solvents, complexing agents, or their combinations, may be used. Suitable solubilizers include propylene glycol, polyethylene glycol 300/400, glycerin, ethanol, polysorbate 80, Cremophor EL, N-methyl-2-pyrrolidone (NMP), meglumine etc.

In various embodiments, the pharmaceutical composition is administered subcutaneously, for example, using single use syringe, prefilled syringe or auto injection or pen device.

In embodiments, the pharmaceutical composition of the present invention is a simple solution consisting of the drug in saline water solution that is pH adjusted to assure the product is within a pH range of 7.5-8.5 throughout its shelf life.

Another embodiment of the present invention is to provide a ready to use stable Pralatrexate composition for subcutaneous administration wherein the composition comprises of pharmaceutically acceptable excipients selected from the group of buffers, pH stabilizers, isotonicity agents, antioxidants, preservatives, and solubility and/or viscosity-enhancing agents.

In another specific embodiments ready to use stable subcutaneous composition of Pralatrexate comprises of at least one osmogen, alkalizer or optionally one surfactant.

In certain embodiments ready to use Pralatrexate composition comprises of specific amount of sodium hydroxide as alkalizer, specific amount of hydrochloric acid as pH adjuster and specific amount of sodium chloride as osmogen.

In certain embodiments ready to use Pralatrexate composition comprises of specific amount of potassium dihydrogen phosphate as buffer, specific amount of hydrochloric acid as pH adjuster and specific amount of sodium chloride as osmogen.

In certain embodiments ready to use Pralatrexate composition comprises of specific amount of sodium hydroxide as alkalizer, specific amount of hydrochloric acid as pH adjuster and specific amount of mannitol as osmogen.

In certain embodiments ready to use Pralatrexate composition comprises of specific amount of sodium hydroxide as alkalizer, specific amount of hydrochloric acid as pH adjuster and specific amount of potassium chloride as osmogen.

In certain embodiments ready to use Pralatrexate composition comprises of specific amount of sodium hydroxide as alkalizer, specific amount of hydrochloric acid as pH adjuster, specific amount of potassium chloride as osmogen and specific amount of Tween 80 or meglumine as surfactant or solubilizer.

In certain embodiments ready to use Pralatrexate composition comprises of specific amount of sodium bicarbonate as buffer and specific amount of hydrochloric acid as pH adjuster.

In certain embodiments ready to use Pralatrexate composition comprises of specific amount of meglumine as surfactant and specific amount of hydrochloric acid as pH adjuster.

Another embodiment of the present invention is to provide ready to use pharmaceutical composition of Pralatrexate wherein the composition is stable over a sufficient time so that the composition has a reasonable shelf life and readily available for use when needed.

According to the present invention ready to use pharmaceutical compositions of Pralatrexate that are stable with respect to pH of the solution and % of API that remains after storage.

Pharmaceutical compositions including Pralatrexate of the present disclosure may include vehicles, carriers, excipients, solvents and the like. Pharmaceutically acceptable vehicles, carriers, excipients, and solvents are known to the skilled practitioner in the pertinent art and can be readily found in Remington's Pharmaceutical Sciences, by E. W. Martin, Mack Publishing Co., Easton, Pa., 15th Edition (1975) and its updated editions, which describes compositions and formulations suitable for pharmaceutical delivery of one or more therapeutic or immunogenic compositions, such as one or more vaccines, and additional pharmaceutical agents. Nonlimiting examples of non-aqueous solvents include propylene glycol, polyethylene glycol, vegetable oils, such as olive oil and canola oil, and injectable organic esters, such as ethyl oleate. Aqueous carriers include water, alcoholic/aqueous solutions, emulsions, or suspensions, including saline and buffered media. Parenteral vehicles include, for example, sodium chloride solution, Ringer's dextrose, dextrose and sodium chloride, lactated Ringer's, or fixed oils. Intravenous vehicles include, for example, fluid and nutrient replenishers, electrolyte replenishers (such as those based on Ringer's dextrose), and the like. Preservatives and other additives may also be present in such compositions and preparations, such as, for example, antimicrobials, antioxidants, chelating agents, colorants, stabilizers, inert gases, and the like.

Some of the compositions may potentially be administered as a pharmaceutically acceptable acid- or base-addition salt, formed by reaction with inorganic acids, such as hydrochloric acid, hydrobromic acid, perchloric acid, nitric acid, thiocyanic acid, sulfuric acid, and phosphoric acid, and organic acids, such as formic acid, acetic acid, propionic acid, glycolic acid, lactic acid, pyruvic acid, oxalic acid, malonic acid, succinic acid, maleic acid, and fumaric acid, or by reaction with an inorganic base such as sodium hydroxide, ammonium hydroxide, potassium hydroxide, and organic bases such as mono-, di-, tri-alkyl and aryl amines and substituted ethanolamines. In some embodiments, the excipient is selected from the group consisting of buffers, pH stabilizers, isotonicity agents, antioxidants, preservatives, and solubility and/or viscosity-enhancing agents.

### FURTHER EMBODIMENTS

In some embodiments, the present disclosure provides ready to use stable pharmaceutical compositions of Pralatrexate for subcutaneous administration.

In some embodiments, the present invention provides ready to use stable pharmaceutical compositions comprising Pralatrexate and one or more pharmaceutically acceptable excipients for subcutaneous administration.

In some embodiments, the present disclosure provides ready to use stable pharmaceutical compositions including at least about 30 mg/ml or at least about 40 mg/ml of Pralatrexate and one or more of alkaliser/solubilizer, surfactant and osmogen for subcutaneous administration.

In some embodiments, the present disclosure provides ready to use stable pharmaceutical compositions including Pralatrexate, or a pharmaceutically acceptable salt, hydrate or ester thereof; and a pharmaceutically acceptable alkaliser/solubilizer, surfactant and osmogen. In some embodiments, the pharmaceutical composition can have a pH in the range of about 7 to about 8.5 and/or can be isoosmotic.

In some embodiments, each mL of ready to use stable Pralatrexate solution includes at least about 30 mg or at least about 40 mg of Pralatrexate, sufficient sodium chloride to achieve an isotonic (280-300 mOsm) solution, and sufficient sodium hydroxide, and hydrochloric acid to adjust and maintain the pH at 7.5-8.5.

In some embodiments, the present disclosure provides ready to use stable concentrated Pralatrexate solutions in aqueous media. In some embodiments, the dose of Pralatrexate is between 40 mg/mL to 100 mg/ml.

In some embodiments, the concentrated ready to use stable solution of Pralatrexate includes 40 mg/mL, 50 mg/mL, 60 mg/mL, 80 mg/ml or 100 mg/ml of Pralatrexate. In an embodiment, the solution includes 60 mg/ml of Pralatrexate.

In some embodiments, the present disclosure provides methods for the preparation of ready to use stable subcutaneous dosage forms of Pralatrexate.

In some embodiments, the present disclosure provides methods for the treatment of peripheral t-cell lymphoma using ready to use subcutaneous dosage forms of Pralatrexate.

The practice of the present invention employs, unless otherwise indicated, conventional techniques of molecular biology (including recombinant techniques), microbiology, cell biology, biochemistry and immunology, which are well within the purview of the skilled artisan. Such techniques are explained fully in the literature, such as, “Molecular Cloning: A Laboratory Manual”, second edition (Sambrook, 1989); “Oligonucleotide Synthesis” (Gait, 1984); “Animal Cell Culture” (Freshney, 1987); “Methods in Enzymology” “Handbook of Experimental Immunology” (Weir, 1996); “Gene Transfer Vectors for Mammalian Cells” (Miller and Calos, 1987); “Current Protocols in Molecular Biology” (Ausubel, 1987); “PCR: The Polymerase Chain Reaction”, (Mullis, 1994); “Current Protocols in Immunology” (Coligan, 1991). These techniques are applicable to the production of the polynucleotides and polypeptides of the invention, and, as such, may be considered in making and practicing the invention. Particularly useful techniques for particular embodiments will be discussed in the sections that follow.

### EXAMPLES

The invention may be further illustrated by the following examples, which are for illustrative purposes only and should not be construed as limiting the scope of the invention in any way. Modification in examples fall within the scope and spirit of appended claims, and which would be obvious to a person skilled in the art based upon the disclosure herein, are also considered to be within the scope of this invention.

### Example-1

Example 1 provides a composition including Pralatrexate formulated for subcutaneous administration. The formulation is described in Table 1, below.

TABLE 1

| | | Quantity (mg/mL) | | |
|---|---|---|---|---|
| S. No | Ingredients | Example 1A | Example 1B | Example 1C |
| 1 | Pralatrexate | 40 | 50 | 60 |
| 2 | Sodium Hydroxide | 7.2 | 10.07 | 8.53 |
| 3 | Hydrochloric Acid | q.s. | q.s. | q.s. |
| 4 | Sodium Chloride | 0.67 | 1.20 | 0.44 |

| | | | |
|---|---|---|---|
| Osmolarity (Osm) | 0.282 | 0.286 | 0.290 |
| pH | 8.06 | 8.12 | 8.1 |

Manufacturing Procedure 40 mg/50 mg/60 mg/mL

Purified water up to 80% of batch volume was dispensed. Sodium hydroxide was added into purified water and stirred for 10 minutes. NaOH and HCl solutions were prepared separately. Pralatrexate was added to sodium hydroxide solution under continuous stirring for 20 minutes. pH of the drug solution was adjusted by adding NaOH/HCl solution to 7.5-8.5 pH. Sodium chloride was added and stirred until dissolved. Final volume was made up using purified water. Finally, the drug solution was filtered under vacuum.

### Example-2 (60 mg/ml)

Example 2 provides a composition including Pralatrexate formulated for subcutaneous administration. The formulation is described in Table 2, below.

TABLE 2

| S. No | Ingredients | Quantity (mg/mL) |
|---|---|---|
| 1 | Pralatrexate | 60 |
| 2 | Potassium Dihydrogen | 6.3 |
| | Phosphate | |
| 3 | Hydrochloric Acid | q.s. |
| 4 | Sodium Chloride | 0.31 |

| | |
|---|---|
| Osmolarity (Osm) | 0.276 |
| pH | 7.98 |

### Example-3 (60 mg/ml)

Example 3 provides a composition including Pralatrexate formulated for subcutaneous administration. The formulation is described in Table 3, below.

TABLE 3

| S. No | Ingredients | Quantity (mg/mL) |
|---|---|---|
| 1 | Pralatrexate | 60 |
| 2 | Sodium Hydroxide | 8.375 |
| 3 | Hydrochloric Acid | q.s. |
| 4 | Mannitol | 8.25 |

| | |
|---|---|
| Osmolarity (Osm) | 0.272 |
| pH | 8.1 |

### Example-4 (60 mg/ml)

Example 4 provides a composition including Pralatrexate formulated for subcutaneous administration. The formulation is described in Table 4, below.

TABLE 4

| S. No | Ingredients | Quantity (mg/mL) |
|---|---|---|
| 1 | Pralatrexate | 60 |
| 2 | Sodium Hydroxide | 8.2375 |
| 3 | Hydrochloric Acid | q.s. |
| 4 | Potassium chloride | 6.25 |

| | |
|---|---|
| Osmolarity (Osm) | 0.295 |
| pH | 8.1 |

### Example-5 (60 mg/ml)

Example 5 provides a composition including Pralatrexate formulated for subcutaneous administration. The formulation is described in Table 5, below.

TABLE 5

| S. No | Ingredients | Quantity (mg/mL) |
|---|---|---|
| 1 | Pralatrexate | 60 |
| 2 | Tween 80 (10%) | 100 |
| 3 | Sodium Hydroxide | 8.25 |
| 4 | Hydrochloric Acid | q.s. |
| 5 | Sodium Chloride | 3.75 |

| | |
|---|---|
| Osmolarity (Osm) | 0.276 |
| pH | 7.98 |

### Example-6 (60 mg/ml)

Example 6 provides a composition including Pralatrexate formulated for subcutaneous administration. The formulation is described in Table 6, below.

TABLE 6

| | | Quantity |
|---|---|---|
| S. No | Ingredients | (mg/mL) |
| 1 | Pralatrexate | 60 |
| 2 | Potassium Hydroxide | 14.0 |
| 3 | Hydrochloric Acid | q.s. |

| | |
|---|---|
| pH | 8.03 |

### Example-7 (60 mg/ml)

Example 7 provides a composition including Pralatrexate formulated for subcutaneous administration. The formulation is described in Table 7, below.

TABLE 7

| | | Quantity |
|---|---|---|
| S. No | Ingredients | (mg/mL) |
| 1 | Pralatrexate | 60 |
| 2 | Sodium Bicarbonate | 30.0 |
| 3 | Hydrochloric Acid | q.s. |
| | pH | 7.98 |

### Example-8 (60 mg/ml)

Example 8 provides a composition including Pralatrexate formulated for subcutaneous administration. The formulation is described in Table 8, below.

TABLE 8

| | | Quantity |
|---|---|---|
| S. No | Ingredients | (mg/mL) |
| 1 | Pralatrexate | 60 |
| 2 | Meglumine | 50.0 |
| 3 | Hydrochloric Acid | q.s. |

| | |
|---|---|
| pH | 8.13 |

### Example-9

Example 9 provides a compositions including Pralatrexate formulated for subcutaneous administration. The formulations are described in Table 9, below.

TABLE 9

| | | Quantity (mg/mL) | |
|---|---|---|---|
| S. No | Ingredients | Example-9A | Example-9B |
| 1 | Pralatrexate | 80 | 100 |
| 2 | Potassium Hydroxide | 38.0 | 44.0 |
| 3 | Hydrochloric Acid 2N | qs | qs |
| 4 | Potassium Hydroxide 2N | qs | qs |
| 5 | Sodium Chloride | qs | qs |

| | | |
|---|---|---|
| pH | 8.03 | 8.09 |

Compositions of Example-2 to Example-9 are manufactured using a similar manufacturing process to that mentioned for Example-1.

### Example-10

Evaluation of plasma pharmacokinetic profile & bioavailability following single dose administration of Pralatrexate Injection 60 mg/ml for Subcutaneous use (Test) and Folotyn (Pralatrexate) injection 20 mg/ml for Intravenous use (Reference) in Healthy Beagle Dogs. The objective of this study is to assess plasma pharmacokinetics profile & bioavailability following single dose administration of Pralatrexate Injection 60 mg/ml for subcutaneous use (TEST) and Folotyn (Pralatrexate) Injection 20 mg/ml for intravenous use (REFERENCE) in healthy beagle dogs.

### Example-11

A Phase 1 randomized, single dose, crossover study was carried out in 16 healthy male and female subjects to evaluate the PK, absolute bioavailability of sub-cutaneous pralatrexate formulation of the present invention. Further, the results are compared with intravenous administration of Folotyn (Pralatrexate) injection.

The bioavailability study was planned in 16 healthy male and female subjects, but only 13 male and female subjects are enrolled into the study. A total of 11 male and female subjects completed the study (all 2 treatments received). Subjects received the following 2 treatments according to the randomization schedule. Subjects have been randomized 1:1 into either Cohort 1 or Cohort 2 (6 subjects in each cohort).
A) Treatment A (Test): Single dose of 5 mg Pralatrexate for subcutaneous injection, 60 mg/ml
B) Treatment B (Reference): Single dose of 5 mg FOLOTYN® (Pralatrexate) injection for intravenous (IV) use, 20 mg/ml.

On the morning of Day 1, subjects in Cohort 1 were administered a single subcutaneous dose of 5 mg Pralatrexate for subcutaneous injection, 60 mg/ml (Treatment A). Similarly, subjects in Cohort 2 were administered a single intravenous dose of 5 mg FOLOTYN® (Pralatrexate) injection for intravenous (IV) use, 20 mg/ml (Treatment B). After 07 day wash out, subjects in Cohort 1 have been administered with 5 mg FOLOTYN® (Pralatrexate) injection for intravenous (IV) use 20 mg/ml (Treatment B); and subjects in Cohort 2 have been administered with 5 mg Pralatrexate for subcutaneous injection, 60 mg/ml (Treatment A).

PK blood samples were collected up to 48 hours post dose on Day 1 and Day 8. Pharmacokinetic and statistical results for Pralatrexate are presented in Table-10A and Table-10B.

TABLE-10A
PK Results for Pralatrexate for Fasting Study (Study No. ABP-00201
(A21.1451))

| | ABP-00201(A21.1451) Pralatrexate | |
|---|---|---|
| PK Parameter | Treatment A (Test) | Treatment B (Reference) |
| Cmax (ng/ml) | 92.385 (±24.3027) | 162.082 (±31.0770) |
| AUC0_24 (hr*ng/mL) | 239.966 (±43.7923) | 162.112 (±39.7510) |
| AUC0-t (hr*ng/ml) | 210.941 (±43.9103) | 135.409 (±38.7295) |
| AUC0-inf (hr*ng/ml) | 325.999 (±111.1564) | 181.188 (±40.0697) |
| Tmax (hr) (median; | 0.8 (0.50-1.50) | 0.33 (0.33-0.37) |
| range) | | |
| Kel (1/hr) | 0.364 (±0.1220) | 0.718 (±0.2020) |
| t½ (hr) | 2.349 (1.33-6.63) | 1.025 (0.59-1.44) |

TABLE 10B
Statistical Results of Pralatrexate (Study No. ABP-00201 (A21.1451))

| | Geometric Mean (A Vs B) | | | 90% | | Intra |
|---|---|---|---|---|---|---|
| PK | Treatment -A | Treatment -B | T/R | Confidence | Power | Subject |
| Parameter | (Test) | (Reference) | Ratio % | Interval | (%) | CV (%) |
| Cₘₐₓ | 90.1177 | 159.5711 | 56.47% | 48.38-65.92 | 78% | 19.9 |
| AUC₀₋ₜ | 206.3456 | 131.7906 | 156.57% | 142.09-172.52 | 98% | 12.4 |
| AUC₀₋₂₄ | 236.0834 | 158.1762 | 149.25% | 137.92-161.52 | 100% | 10.1 |
| AUC_{0-inf} | 308.9439 | 177.5529 | 174.00% | 146.21-207.07 | 70% | 19.9 |

Mean Subcutaneous Absolute Bioavailability (F)- Pralatrexate

| | |
|---|---|
| F (For | 1.8278 |
| Treatment -A) | |

### Example-12

A Phase II, Prospective, Randomized, Open-Label, Multicenter, Dose-Finding Study to Evaluate the Pharmacokinetics of Subcutaneous Pralatrexate in Patients with Relapsed/Refractory Peripheral T-Cell Lymphoma (R/R PTCL).

The objective of the above protocol is to evaluate the Pharmacokinetic/Pharmacodynamic (PK/PD) of pralatrexate in different subcutaneous (SC) regimens. Patients were randomized into three groups. Group A patients will be administered SC pralatrexate dose 13 mg/m² weekly for two weeks (Day 1 and Day 8) in a cycle of 21 days. Group B will be administered Pralatrexate dose SC of 20 mg/m² weekly for two weeks (Day 1 and Day 8) in a cycle of 21 days and Group C patients will be administered Pralatrexate dose SC of 30 mg/m² weekly for two weeks (Day 1 and Day 8) in a cycle of 21 days.

### OTHER EMBODIMENTS

From the foregoing description, it will be apparent that variations and modifications may be made to the invention described herein to adopt it to various usages and conditions. Such embodiments are also within the scope of the following claims.

The recitation of a listing of elements in any definition of a variable herein includes definitions of that variable as any single element or combination (or subcombination) of listed elements. The recitation of an embodiment herein includes that embodiment as any single embodiment or in combination with any other embodiments or portions thereof.

All patents and publications mentioned in this specification are herein incorporated by reference to the same extent as if each independent patent and publication was specifically and individually indicated to be incorporated by reference.

What is claimed is:

1. A pharmaceutical composition comprising Pralatrexate or a pharmaceutically acceptable salt, hydrate or ester thereof and a pharmaceutically acceptable excipient, wherein the pharmaceutical composition is formulated for subcutaneous administration.

2. The pharmaceutical composition of claim 1 comprising at least about 30 mg/ml of Pralatrexate.

3. The pharmaceutical composition of claim 1 or 2, comprising at most about 100 mg/ml Pralatrexate.

4. The pharmaceutical composition of any one of claims 1 to 3, wherein the composition comprises from about 30 mg/ml to 90 mg/ml Pralatrexate, from about 30 mg/ml to about 80 mg/ml Pralatrexate, from about 30 mg/ml to about 70 mg/ml Pralatrexate, from about 35 mg/ml to 90 mg/ml Pralatrexate, from about 35 mg/ml to about 80 mg/ml Pralatrexate, from about 35 mg/ml to about 70 mg/ml Pralatrexate, from about 40 mg/ml to 90 mg/ml Pralatrexate, from about 40 mg/ml to about 80 mg/ml Pralatrexate, or from about 40 mg/ml to about 70 mg/ml Pralatrexate.

5. The pharmaceutical composition of any one of claims 1 to 4, wherein the composition comprises about 40 mg/ml Pralatrexate, about 50 mg/ml Pralatrexate, about 60 mg/ml Pralatrexate, about 70 mg/ml Pralatrexate, or about 80 mg/ml Pralatrexate.

6. The pharmaceutical composition of any one of claims 1 to 4, wherein the composition comprises about 60 mg/ml Pralatrexate.

7. The pharmaceutical composition of any one of claims 1-6, wherein the pharmaceutically acceptable excipient comprises a pharmaceutically acceptable alkalizer and/or solubilizer and/or surfactant and/or osmogen.

8. The pharmaceutical composition of any one of claims 1-7, wherein the pharmaceutically acceptable excipient comprises one or more of a buffer, a pH stabilizer, an isotonicity agent, an antioxidant, a preservative, a solubility-enhancing agent, and a viscosity-enhancing agent.

9. The pharmaceutical composition of any one of claims 1-8, wherein the pharmaceutical composition includes an alkalizer and the alkalizer comprises lithium hydroxide, sodium hydroxide, potassium hydroxide or calcium hydroxide.

10. The pharmaceutical composition of any one of claims 1-9, wherein the pharmaceutical composition comprises a solubilizer and the solubilizer comprises one or more of propylene glycol, polyethylene glycol 300/400, glycerin, ethanol, polysorbate 80, Cremophor EL, N-methyl-2-pyrrolidone (NMP), or meglumine.

11. The pharmaceutical composition of any one of claims 1-10, wherein the pharmaceutical composition comprises a surfactant and the surfactant comprises Tween 80 or meglumine.

12. The pharmaceutical composition of any one of claims 1-11, wherein the pharmaceutical composition comprises an osmogen, and the osmogen comprises one or more of potassium chloride, sodium chloride, or mannitol.

13. The pharmaceutical composition of any one of claims 1-12, wherein the composition has an osmolarity of between about 250 mOsm to about 350 mOsm.

14. The pharmaceutical composition of any one of claims 1-13, wherein the composition has greater bioavailability of pralatrexate when administered to a subject as compared to an intravenous formulation of Pralatrexate.

15. The pharmaceutical composition of claim 14, wherein the composition has at least 10% greater bioavailability of Pralatrexate as compared to an intravenous formulation of Pralatrexate.

16. The pharmaceutical composition of claim 14, wherein the composition has at least 20% greater bioavailability of Pralatrexate as compared to an intravenous formulation of Pralatrexate.

17. The pharmaceutical composition of claim 14, wherein the composition has at least 30% greater bioavailability of Pralatrexate as compared to an intravenous formulation of Pralatrexate.

18. The pharmaceutical composition of any one of claims 1-17, wherein a single dose subcutaneous administration of the composition to a subject provides at least one of:
a) a Cmax of from 60 to 120 ng/ml,
b) an AUC0→τ of from 150 to 270 hr*ng/ml,
c) an AUC0→24 of from 180 to 300 hr*ng/ml,
d) an AUC0→inf of from 150 to 400 hr*ng/ml,
e) a Tmax of from 0.5-1.5 hr,
f), a Kel of from 0.2-0.4 l/hr,
g) a t1/2 of from 1.3-6.6 hr, and
h) a mean subcutaneous absolute bioavailability of from 1.7-2.1.

19. The pharmaceutical composition according to any one of claims 1-18, wherein a single dose of subcutaneous administration of the composition to a subject provides a Cₘₐₓ of from 60 to 120 ng/ml and a Tₘₐₓ of from 0.5-1.5 hr.

20. The pharmaceutical composition according to any one of claims 1-19, wherein a single dose of subcutaneous administration of the composition to a subject provides an AUC0→24 of from 180 to 300 hr*ng/mL.

21. The pharmaceutical composition according to any one of claims 1-20, wherein said pharmaceutical composition comprises:
a) from about 30 mg/ml to about 110 mg/ml Pralatrexate;
b) a pH adjuster;
c) optionally from about 5 mg/ml to about 20 mg/ml alkalizer,
d) optionally from about 5 mg/ml to about 35 mg/ml buffer;
e) optionally from about 0.2 mg/ml to about 10 mg/ml osmogene or from about 6 to about 10 mg/ml non-inorganic salt osmogene; and
f) optionally from about 40 mg/ml to about 110 mg/ml surfactant.

22. The pharmaceutical composition according to any one of claims 1-21, wherein said pharmaceutical composition comprises:
a) from about 30 mg/ml to about 110 mg/ml Pralatrexate;
b) hydrochloric acid;
c) optionally from about 5 mg/ml to about 20 mg/ml sodium hydroxide,
d) optionally from about 5 mg/ml to about 35 mg/ml sodium bicarbonate, potassium dihydrogen phosphate, or a combination thereof;
e) optionally from about 0.2 mg/ml to about 10 mg/ml osmogen selected from sodium chloride, potassium chloride, mannitol or from about 6 to about 10 mg/ml mannitol; and
f) optionally from about 40 mg/ml to about 110 mg/ml surfactant of a surfactant selected from meglumine and Tween 80.

23. The pharmaceutical composition according to any one of claims 1-22, wherein the pharmaceutical composition is isosmotic.

24. The pharmaceutical composition according to any one of claims 1-23, wherein the pharmaceutical composition has a pH between about 7 to about 8.5.

25. A method for the treatment of lymphoma comprising administering to a subject in need thereof a pharmaceutical composition subcutaneously to the subject, wherein the pharmaceutical composition comprises Pralatrexate and a pharmaceutically acceptable excipient.

26. The method according to claim 25, wherein the lymphoma is peripheral T-cell lymphoma (PTCL).

27. The method of claim 25 or 26, wherein the pharmaceutical composition comprises at least about 30 mg/ml of Pralatrexate.

28. The method of any one of claims 25-27, wherein the pharmaceutical composition comprises at most 100 mg/ml.

29. The method of any one of claims 25-28, wherein the pharmaceutical composition comprises from about 30 mg/ml to 90 mg/ml Pralatrexate, from about 30 mg/ml to about 80 mg/ml Pralatrexate, from about 30 mg/ml to about 70 mg/ml Pralatrexate, from about 40 mg/ml to 90 mg/ml Pralatrexate, from about 40 mg/ml to about 80 mg/ml Pralatrexate, or from about 40 mg/ml to about 70 mg/ml Pralatrexate.

30. The method of any one of claims 25-29, wherein the composition comprises about 60 mg/ml Pralatrexate.

31. The method of any one of claims 25-30, wherein the pharmaceutical composition further comprises a pharmaceutically acceptable alkalizer and/or solubilizer and/or surfactant and/or osmogen.

32. The method of any one of claims 25-31, wherein the pharmaceutical composition includes an alkalizer and the alkalizer comprises lithium hydroxide, sodium hydroxide, potassium hydroxide or calcium hydroxide.

33. The method of any one of claims 25-32, wherein the pharmaceutical composition comprises a solubilizer and the solubilizer comprises one or more of propylene glycol, polyethylene glycol 300/400, glycerin, ethanol, polysorbate 80, Cremophor EL, N-methyl-2-pyrrolidone (NMP), or meglumine.

34. The method of any one of claims 25-33, wherein the solubilizer is one or more of propylene glycol, polyethylene glycol 300/400, glycerin, ethanol, polysorbate 80, Cremophor EL, N-methyl-2-pyrrolidone (NMP), or meglumine.

35. The method of any one of claims 25-34, wherein the pharmaceutical composition comprises a surfactant and the surfactant comprises Tween 80 or meglumine.

36. The method of any one of claims 25-35, wherein the pharmaceutical composition comprises an osmogen, and the osmogen comprises one or more of potassium chloride, sodium chloride, or mannitol.

37. The method of any one of claims 25-36, wherein the composition has an osmolarity of between about 250 mOsm to about 350 mOsm.

38. The method of any one of claims 25-37, wherein the administering causes greater bioavailability of Pralatrexate in the subject, as compared to administering an intravenous formulation of Pralatrexate (e.g., as determined by comparing AUC0→τ, AUC0→24, or AUC0→inf values following administration of intravenous or subcutaneous injections of the same weight of Pralatrexate such as 5 mg Pralatrexate).

39. The method of claim 38, wherein the administering causes at least 10% greater bioavailability of Pralatrexate in the subject, as compared to administering an intravenous formulation of Pralatrexate.

40. The method of claim 38, wherein the administering causes at least 20% greater bioavailability of Pralatrexate in the subject, as compared to administering an intravenous formulation of Pralatrexate.

41. The method of claim 38, wherein the administering causes at least 30% greater bioavailability of Pralatrexate in the subject, as compared to administering an intravenous formulation of Pralatrexate.

42. The method of any one of claims 25-41, wherein a single dose of administering to the subject provides at least one of:
a) a Cmax of from 60 to 120 ng/ml,
b) an AUC0→τ of from 150 to 270 hr*ng/ml,
c) an AUC0→24 of from 180 to 300 hr*ng/ml, and
d) an AUC0→inf of from 150 to 400 hr*ng/mL,
e) a Tmax of from 0.5-1.5,
f), a Kel of from 0.2-0.4 l/hr,
g) a t1/2 of from 1.3-6.6 hr, and
h) a mean subcutaneous absolute bioavailability of from 1.7-2.1.

43. The method according to any one of claims 25-42, wherein a single dose of subcutaneous administration to the subject provides a Cₘₐₓ of from 60 to 120 ng/ml and a Tₘₐₓ of from 0.5-1.5 hr.

44. The pharmaceutical composition according to any one of claims 25-43, wherein a single dose of subcutaneous administration to the subject provides an AUC0→24 of from 180 to 300 hr*ng/mL.

45. The method according to any one of claims 25-44, wherein the pharmaceutical composition comprises:
a) from about 30 mg/ml to about 110 mg/ml pralatrexate;
b) a pH adjuster (e.g., hydrochloric acid);
c) optionally from about 5 mg/ml to about 20 mg/ml alkalizer (e.g., sodium hydroxide,
d) optionally from about 5 mg/ml to about 35 mg/ml buffer (e.g., sodium bicarbonate, potassium dihydrogen phosphate);
e) optionally from about 0.2 mg/ml to about 10 mg/ml osmogene (e.g., sodium chloride, potassium chloride, mannitol) or from about 6 to about 10 mg/ml non-inorganic salt osmogene (e.g., mannitol); and
f) optionally from about 40 mg/ml to about 110 mg/ml surfactant (e.g., meglumine, Tween 80).

46. The method according to any one of claims 25-45, wherein the pharmaceutical composition comprises:
a) from about 30 mg/ml to about 110 mg/ml pralatrexate;
b) hydrochloric acid;
c) optionally from about 5 mg/ml to about 20 mg/ml sodium hydroxide,
d) optionally from about 5 mg/ml to about 35 mg/ml buffer selected from sodium bicarbonate, potassium dihydrogen phosphate, and combinations thereof;
e) optionally from about 0.2 mg/ml to about 10 mg/ml osmogene selected from sodium chloride, potassium chloride, mannitol or from about 6 to about 10 mg/ml mannitol; and
f) optionally from about 40 mg/ml to about 110 mg/ml surfactant selected from meglumine, Tween 80, and combinations thereof.

47. The method according to any one of claims 25-46, wherein the pharmaceutical composition is isosmotic.

48. The method according to any one of claims 25-47, wherein the pharmaceutical composition has a pH between about 7 to about 8.5.

49. The method according to any one of claims 25-48, wherein the pharmaceutical composition is administered weekly.

50. The method according to any one of claims 25-49, wherein the pharmaceutical composition is administered weekly for two weeks in a 21-day administration cycle.

51. The method according to any one of claims 28-50, wherein the pharmaceutical composition comprises from about 10 mg/m² to 40 mg/m².

52. The method according to any one of claims 28-50, wherein the pharmaceutical composition comprises from about 13 mg/m², about 20 mg/m², or about 30 mg/m² Pralatrexate.
